(19) United States
(12) Patent Application Publication
(10) Pub. No.: US 2019/0192406 A1
(43) Pub. Date: Jun. 27, 2019

(54) THICKENED FORMULATIONS CONTAINING OXIDIZING AGENTS IN PACKAGES FORMED FROM BARRIER-LAYERED FOILS

(71) Applicant: Henkel AG & Co. KGaA, Duesseldorf (DE)

(72) Inventors: Marc Nowottny, Moenchengladbach (DE); Burkhard Mueller, Duesseldorf (DE)

(73) Assignee: Henkel AG & Co. KGaA, Duesseldorf (DE)

(21) Appl. No.: 16/331,898

(22) PCT Filed: Jun. 30, 2017
(86) PCT No.: PCT/EP2017/066249

§ 371 Date: Mar. 8, 2019

(30) Foreign Application Priority Data
Sep. 9, 2016 (DE) 10 2016 217 177.1

(51) Int. Cl. A61K 8/49; A61Q 5/08; A61Q 5/10; A61K 8/81; A61K 8/46; A61K 8/22; A61K 8/58; A61K 8/44; B65D 75/28; B65D 65/40

(52) U.S. Cl. CPC A61K 8/4926; A61K 2800/48; A61Q 5/10; A61K 8/8152; A61K 8/8147; A61K 8/463; A61K 8/22; A61K 8/58; A61K 8/44; B65D 75/28; B65D 65/40; A61K 2800/4322; A61K 2800/4324; A61K 2800/87; A61Q 5/08

(57) ABSTRACT

The present disclosure concerns a cosmetic product for changing the natural colour of keratinous fibres, in particular human hair, which contains at least one package (VP) as well as a cosmetic composition (KM) contained in this package (VP). The package is formed from a multilayer foil (F) which contains at least two polymer layers (P1) and (P2) as well as at least one barrier layer (BS). The cosmetic composition comprises at least one oxidizing agent, at least one thickening agent as well as at least one anionic surfactant. Using the package (VP) in combination with the cosmetic composition (KM) surprisingly does not result in expansion of the package or excess loss of water from the medium (KM) during storage.

### CROSS-REFERENCE TO RELATED APPLICATION

This application is a U.S. National-Stage entry under 35 U.S.C. § 371 based on International Application No. PCT/EP2017/066249, filed Jun. 30, 2017, which was published under PCT Article 21(2) and which claims priority to German Application No. 10 2016 217 177.1, filed Sep. 9, 2016, which are all hereby incorporated in their entirety by reference.

### TECHNICAL FIELD

The present disclosure pertains to the field of cosmetics and relates to a product for the oxidative coloration of keratinous fibres, in particular human hair, which comprises an oxidizing agent-containing composition packaged in a package. The oxidizing agent-containing composition contains specific thickening agents as well as anionic surfactants. The package is a package prepared from a special multi-layered laminated foil system the wall of which comprises at least two polymer layers and a barrier layer. In this regard, the barrier layer acts to block the passage of gases and water vapour.

### BACKGROUND

Changing the colour of keratinous fibres, in particular hair, constitutes an important area of modern cosmetics. By way of this, the appearance of the hair as well as the latest fashion trends and also an individual's aspirations can be harmonized. In order to change the hair colour, the person skilled in the art will be aware of a variety of possibilities.

The colour of hair can be temporarily changed using direct dyes. Here, fully-formed dyes diffuse out of the colorant into the hair fibres. Colouring with direct dyes is associated with little damage to the hair; a disadvantage, however, is that the colours obtained with direct dyes do not last as long and wash out more quickly.

If the consumer wants a long-lasting colour result or a nuance which is lighter than the initial colour of the hair, then oxidative colour-changing agents are usually employed. For permanent, intensive colours with appropriate fastness, what are known as oxidative dyes are used. Colorants of this type usually contain oxidative dye precursors, what are known as developer components and coupler components, which together form the actual dyes under the influence of oxidizing agents—usually hydrogen peroxide. Oxidative dyes are exemplified by excellent, long-lasting colour results.

Lightening or bleaching of hair by itself is often carried out using oxidizing agents without the addition of oxidative dye precursors. For a medium bleaching effect, hydrogen peroxide alone is sufficient for use as the oxidizing agent; to obtain a stronger bleaching effect, a mixture of hydrogen peroxide and peroxydisulphate salts is usually employed.

Oxidative colour changing agents are usually offered for sale in the form of two-component agents in which two different preparations are packaged separately in two separate packages and which are only mixed together just before use.

The first preparation is a formulation—which is usually acidic for reasons of stability—which, for example, contains hydrogen peroxide as the oxidizing agent, in concentrations of from about 1.5 to about 12% by weight. The oxidizing agent formulation is usually in the form of an emulsion or dispersion and as a rule is in a plastic bottle (developer bottle) provided with a reclosable dispensing opening.

This oxidizing agent formulation is mixed with a second preparation prior to use. This second preparation is a formulation which is alkaline, which is often in the form of a cream or a gel and which, when a change of colour is desired along with lightening, additionally contains at least one oxidative dye precursor. This second preparation may, for example, be in the form of a tube or in the form of a plastic or glass container.

In the case of the usual application form described above, the second preparation, which contains the alkalizing agent and/or the oxidative dye precursors, is transferred from the tube or container into the developer bottle and then mixed with the hydrogen peroxide preparation already in the developer bottle by shaking. In this manner, the ready-to-use mixture is produced in the developer bottle. Application to the hair is then carried out via a small nozzle or dispensing opening at the top of the developer bottle. The nozzle or dispensing opening is opened after shaking and the ready-to-use mixture can be dispensed by squeezing the flexible developer bottle.

Using the developer bottle requires the user to have a certain amount of skill, and so some users prefer to produce the ready-to-use mixture in a mixing bowl and to apply it using a brush.

When producing the ready-to-use mixture in a bowl, both components—the first preparation which contains the oxidizing agent and the second preparation with the alkalizing agent and/or the oxidative dye precursors—are transferred in their entirety into a bowl or similar vessel and are then stirred in it, for example with the aid of a brush. The ready-to-use mixture is then removed from the mixing bowl using the brush. In this form of application, it is not necessary to use a bulky and expensive developer bottle, and research is still being carried out into inexpensive forms of packaging for the oxidizing agent preparation that do not use much material.

Packaging in the form of pouches or bags, which as a rule are prepared from plastic foils or from metal foils, are candidates for inexpensive forms of packaging which do not consume much material.

Packaging of this type may, for example, be produced by bonding or hot pressing two plastic foils placed one on top of one another, wherein bonding is carried out at all edges of the foil. The interior space of the package (i.e. the plastic bag) produced by the bonding can then be filled with the desired cosmetic preparation. The package can be opened by tearing or cutting the plastic bag.

However, filling packages of this type with oxidizing agent preparations is fraught with problems which are caused by the reactivity of the oxidizing agent. Oxidizing agents are highly reactive substances which—independently of the storage conditions or of the possible presence of impurities which cause decomposition—can partially decompose, with the concomitant formation of oxygen (i.e. of gas).

As a rule, the interior volume of developer bottles which are known in the prior art are filled with the oxidizing agent composition to the half-way mark at most, but are usually only one-third filled. As a rule, developer bottles are produced from polyethylene. Because polyethylene is permeable to water vapour as well as to gases, no extra pressure or only a slight overpressure occurs in the developer bottle. Furthermore, developer bottles are usually provided with stable, thick walls and a stable screw closure, so that the diffusion of water vapour or gases through the thickness of the walls is reduced and a small increase in pressure inside the bottle does not have any negative effects.

In contrast, however, bag-like packages are usually completely filled with the liquid preparation, and in the filled bag there is almost no head of air. In addition, packages of this type should be flexible, and upon opening (for example tearing or cutting), the preparation should be dispensed in an uncontrolled manner. For this reason, when packaging liquid preparations, the occurrence of excess pressure in the packaging should be avoided if possible.

Thus, if an oxidizing agent composition is packaged in a package of this type, then the gas (oxygen) which is developed during storage may result in expansion of the packaging. Because the edges of the packaging are usually only bonded, then in the worst case scenario, substantial expansion may lead to bursting of the package. For these reasons, when storing compositions containing oxidizing agents, the choice of foil material from which the package is formed is of vital importance.

Packages which consist purely of plastic such as polyethylene or polypropylene are permeable to both water vapour and to gases. Thus, when storing a preparation containing oxidizing agents in a package formed from polyethylene or polypropylene, the package does not expand. Because of the high permeability of the comparatively thin foil of the package as regards water vapour, however, the water content of the preparation is reduced. If the preparation in the packaging is stored for several weeks or months, the loss of water exceeds the permissible maximum value for sufficient stability on storage.

Completely airtight packages are produced, for example, from plastic foils which are laminated with a metal layer, for example with an aluminium layer. These packages are impermeable to water vapour and gases. If these packages are filled with a preparation containing an oxidizing agent, then the gas released during decomposition of the oxidizing agent cannot escape, and the packaging will expand as described above and might burst.

### BRIEF SUMMARY

Cosmetic products for changing the natural colour of keratinous fibres are provided herein. In an embodiment, a cosmetic product for changing the natural colour of keratinous fibres includes:

(i) a package, including a multilayer foil, which includes a first polymer layer, a second polymer layer, and a barrier layer, and

(ii) a cosmetic composition, which is packaged in the package and includes:
a) an oxidizing compound,
b) a thickening agent selected from the group of copolymers of (meth)acrylic acid and (meth)acrylic acid esters, copolymers of (meth)acrylates and (meth)acrylamides, copolymers of hydroxyethyl(meth)acrylates and (meth)acrylamides, copolymers of (meth)acrylates, (meth)acrylamides and ethoxylated (meth)acrylic acid esters as well as mixtures thereof, and
c) an anionic surfactant.

In another embodiment, a cosmetic product for changing the natural colour of keratinous fibres includes:

(i) a package, including a multilayer foil, which includes:
a first polymer layer including polypropylene and having a layer thickness of from about 60.0 μm to about 90.0 μm,
a second polymer layer including polyethylene terephthalate and having a layer thickness of from about 10.0 μm to about 20.0 μm, and
a barrier layer including:
   a layer including silicon dioxide; and
   a layer including an inorganic-organic hybrid polymer;
wherein the barrier layer has a layer thickness of from about 10.0 nm to 150 nm and is disposed between the first polymer layer and the second polymer layer, and

(ii) a cosmetic composition, which is packaged in the package and includes:
a) an oxidizing compound including hydrogen peroxide and present in a total quantity of from about 1.5 to about 15% by weight, with respect to the total weight of the cosmetic composition,
b) a thickening agent including a copolymer of (meth)acrylic acid and acrylic acid ethyl ester and present in an amount of from about 0.40 to about 4.5% by weight, with respect to the total weight of the cosmetic composition;
c) an anionic surfactant including a sodium salt of C₁₂-C₁₄ alkylether sulphate with about 2 mol of ethylene oxide per mol of C₁₂-C₁₄ alkylether sulphate, wherein the anionic surfactant is present in a total quantity of from about 0.27 to about 1.0% by weight, with respect to the total weight of the cosmetic composition;
d) water present in a total quantity of from about 60 to about 97% by weight, with respect to the total weight of the cosmetic composition.

In another embodiment, a cosmetic product for changing the natural colour of keratinous fibres includes:

(i) a package, including a multilayer foil, which includes:
a first polymer layer including polypropylene and having a layer thickness of from about 60.0 μm to about 90.0 μm,
a second polymer layer including polyethylene terephthalate and having a layer thickness of from about 10.0 μm to about 20.0 μm, and
a barrier layer including:
   a layer including silicon dioxide; and
   a layer including an inorganic-organic hybrid polymer;
wherein the barrier layer has a layer thickness of from about 10.0 nm to 150 nm and is disposed between the first polymer layer and the second polymer layer, and

(ii) a cosmetic composition, which is packaged in the package and consists of:
a) an oxidizing compound,
b) a thickening agent including a copolymer of (meth)acrylic acid and acrylic acid ethyl ester;
c) an anionic surfactant including a sodium salt of C₁₂-C₁₄ alkylether sulphate with about 2 mol of ethylene oxide per mol of C₁₂-C₁₄ alkylether sulphate;
d) water present in a total quantity of from about 60 to about 97% by weight, with respect to the total weight of the cosmetic composition;
e) an inorganic alkalizing agent; and
f) a mixture of acids including:
at least one of dipicolinic acid, disodium pyrophosphate, ethylenediamine tetraacetic acid, or salts thereof; and
1-hydroxyethane-1,1-diphosphonic acid.

### DETAILED DESCRIPTION

The following detailed description is merely exemplary in nature and is not intended to limit the disclosure or the application and uses of the subject matter as described herein. Furthermore, there is no intention to be bound by any theory presented in the preceding background or the following detailed description.

The aim of the present application was therefore to package the oxidizing agent composition inexpensively, in a manner which is economical with material, which saves on space and is safe, and in particular is stable upon storage.

Surprisingly, it has now been shown that compositions containing oxidizing agents can be packaged in a manner which is stable upon storage if firstly, special packages are used which include special foil laminate systems and which additionally have a barrier layer. Secondly, the oxidizing agent preparation has to be thickened with special thickening agents.

The subject matter of the present disclosure is a cosmetic product for changing the natural colour of keratinous fibres, in particular human hair, comprising:

(i) at least one package (VP), comprising at least one multilayer foil (F), which contains at least one first polymer layer (P1), at least one second polymer layer (P2), as well as at least one barrier layer (BS), and
(ii) at least one cosmetic composition (KM), which is packaged in the package (VP) and contains:
a) at least one oxidizing compound,
b) at least one thickening agent selected from the group formed by copolymers of (meth)acrylic acid and (meth)acrylic acid esters, copolymers of (meth)acrylates and (meth)acrylamides, copolymers of hydroxyethyl(meth)acrylates and (meth)acrylamides, copolymers of (meth)acrylates, (meth)acrylamides and ethoxylated (meth)acrylic acid esters as well as mixtures thereof, in particular copolymers of (meth)acrylic acid and acrylic acid ethyl esters, and
c) at least one anionic surfactant.

The term “keratinous fibres”, “keratin-containing fibres” or keratin fibres” should be understood to mean fur, wool, feathers and in particular human hair. Although the agent as contemplated herein is primarily suitable for lightening and colouring keratinous fibres, in principle there is no impediment to using it in other areas.

The product as contemplated herein is a product for the oxidative coloration of keratinous fibres, i.e. a product which is used on the human head in order to carry out an oxidative coloration, lightening, bleaching, fading or nuancing of the hair. In this context, the term “nuancing” should be understood to mean a coloration in which the colour result is lighter than the initial colour of the hair.

Furthermore, the term “package” as used in the context of the present disclosure should be understood to mean a package which is preferably in the form of a sachet. A sachet is a small package in the form of a pouch or bag which is often used when packaging cosmetics. The capacity of the package, in particular of the sachet, may, for example, be from about 5 to about 1000 mL, preferably from about 10 to about 200 mL and particularly preferably from about 20 to about 50 mL.

In addition, in the context of the present disclosure, the term “multilayer foil” (F) should be understood to mean a thin, flat web that is capable of being rolled up, formed from the at least one polymer layer (P1) and the at least one polymer layer (P2). This multilayer foil (F) forms the wall of the package (VP). The polymer layers (P1) and (P2) preferably comprise polymers which are capable of forming films. Furthermore, the polymer layers (P1) and (P2) are preferably polymer layers which differ from one another. The package additionally contains a barrier layer (BS) which prevents or reduces the passage of water vapour and other gases such as oxygen, for example, i.e. prevents or reduces the diffusion of these gases through the wall of the package.

Furthermore, the term “thickening agent” as used in the context of the present disclosure should be understood to mean compounds which can bind liquids, in particular water, and increase the viscosity of these liquids. In the context of the present disclosure, this also includes gel-forming agents which are capable of thickening liquids into compositions with a gel-like consistency or into gels. The term “gel-like cosmetic agents or gels” as used as contemplated herein should be understood to mean dimensionally stable, readily deformable disperse systems formed from at least two components, the gel-forming agent (usually a solid, colloidally disperse substance with long or highly-branched compounds) and a liquid (usually water) as the dispersing agent. In the liquid, the gel-forming agent forms a three-dimensional network, wherein the individual gel-forming compounds adhere together by employing primary and/or secondary valency bonding to various points in space. In this context, the term “(meth)acrylate” should be understood to mean both methacrylates as well as acrylates. Examples of suitable thickening agents are the thickening agents which are available under the INCI names acrylates copolymer, sodium acrylate/sodium acryloyldimethyl taurate copolymer, hydroxyethyl acrylate sodium acryloyldimethyl taurate copolymer, polyacrylate crosspolymer 6, ammonium acryloyl dimethyl taurate (NH₄AMPS), dimethyl acrylamide, lauryl methacrylate as well as laureth-4 methacrylate.

The term “anionic surfactant” as used as contemplated herein should be understood to mean amphiphilic (bifunctional) compounds which include at least one hydrophobic and at least one hydrophilic molecular portion. The hydrophobic residue is preferably a hydrocarbon chain containing from about 8 to about 28 carbon atoms which may be saturated or unsaturated, linear or branched. Particularly preferably, this C₈-C₂₈ alkyl chain is linear. In addition, these surfactants contain at least one carboxylate and/or sulphonate group.

The cosmetic product as contemplated herein comprises, as the first component, a package (VP) which comprises at least one multilayer foil (F). This foil contains at least one first polymer layer (P1), at least one second polymer layer (P2) as well as at least one barrier layer (BS). This multilayer foil constitutes the wall or the outer sheath of the package. As described above, a package of this type is usually produced by bonding, pressing or welding two superimposed pieces of foil (wherein the package (VP) is simultaneously filled with the cosmetic composition (KM)), i.e. all of the edges of a package of this type are sealed. This package can be opened, for example, by tearing or cutting it open.

The thickness of the multilayer foil (F) should in this regard be fixed in a manner such that it has sufficient mechanical stability, but at the same time the foil (F)—and thus the package (VP) produced from the foil—should be flexible enough to enable the entirety of the cosmetic composition (KM) to be removed from the opened package (VP) by compression or squeezing. These requirements are satisfied in particular if the foil (F) has a specific total thickness. Preferred embodiments of the present disclosure are thus exemplified in that the at least one multilayer foil has a total thickness of from about 21 μm to about 2.0 mm, preferably of from about 30 μm to about 1.0 mm, preferably of from about 50 μm to about 500 μm, in particular of from about 60 μm to about 200 μm. The term “total thickness of the foil (F)” as used in the context of the present disclosure should be understood to mean the sum of the thicknesses of all of the individual layers constituting the foil (F).

The order of the layers (P1), (P2) as well as (BS) within the multilayer foil (F) may differ. Furthermore, it is also possible for the foil (F) to comprise other layers in addition to the layers mentioned above. In addition, as contemplated herein, it is advantageous for all of the layers which have been mentioned to be respectively orientated parallel to the surfaces of the foil (F), meaning that all of the layers have the same orientation.

As contemplated herein, particularly preferably, the barrier layer (BS) is disposed on the side which comes into contact with the cosmetic composition (KM). The first polymer layer (P1) then adjoins the barrier layer (BS) on the one hand and the second polymer layer (P2), which is on the outside of the package, on the other hand. The polymer layer (P1) here is different from the polymer layer (P2). Here, the barrier layer (BS) acts as a support layer onto which the first polymer layer (P1) is applied. Next, the second polymer layer (P2) is applied to this polymer layer (P1). The three layers (BS), (P1) and (P2) together form a foil (F) which preferably has a total thickness of from about 30 μm to about 1.0 mm.

In the context of the present disclosure, however, an arrangement in which the barrier layer (BS) is between the first polymer layer (P1) and the second polymer layer (P2) is particularly preferred. In this case, the multilayer foil (F) includes three layers, wherein the layer (P1) is innermost and comes into contact with the cosmetic composition (KM). The layer (P1) comes into contact with the barrier layer (BS), and the barrier layer (BS) in turn comes into contact with the layer (P2). In this lay-up, the layers (P1) and (P2) do not adjoin each other, but are separated by the barrier layer (BS). In this arrangement, the layers (P1) and (P2) may in principle be formed from the same polymeric material, however preferably, the two layers (P1) and (P2) include different polymeric materials. The three layers (P1), (BS) and (P2) together form a foil (F) with a total thickness which is preferably from about 30 μm to about 1.0 mm. The particular advantage of this arrangement is that the—frequently very thin—barrier layer (BS) is neither on the inner nor on the outer surface of the multilayer foil (F), but is protected in the direction of the interior by the polymer layer (P1) and in the direction of the exterior by the polymer layer (P2). In this manner, in this arrangement, mechanical abrasion or mechanical destruction of the barrier layer (BS) can be avoided as far as possible. Thus, in the context of the present disclosure, it is possible for the at least one multilayer foil (F) to contain the at least one barrier layer (BS) between the at least one first polymer layer (P1) and the at least one second polymer layer (P2). The use of packages of this type has been shown to be particularly advantageous having regard to increased stability upon storage, because this arrangement exhibits neither expansion nor delamination upon longer contact time with a composition containing an oxidizing agent.

As contemplated herein, a foil (F) in which the first polymer layer (P1) is disposed on the side which comes into contact with the cosmetic composition (KM) is also particularly preferred. The second polymer layer (P2) adjoins the polymer layer (P1) and is different therefrom. The barrier layer (BS) is on the outside. In foils (F) with this type of lay-up, the layer (P1) may act as the polymer support layer, for example, onto which the second polymer layer (P2) is then applied. Next, the side which adjoins (P2) (i.e. the outside) is provided with the barrier layer. Thus, in the context of the present disclosure, it is advantageous for the at least one multilayer foil (F) to have the at least one barrier layer (BS) on the outside of the package (VP). As contemplated herein, the “outside” of the package (VP) should be understood to mean that side of the package which does not come into contact with the cosmetic composition (KM), but which comes into contact with the environment. In this manner, the three layers (P1), (P2) and (BS) form a foil (F) which preferably has a total thickness of from about 30 μm to about 1.0 mm. The use of packages of this type has been shown to be particularly advantageous as regards increasing the stability on storage, because this arrangement exhibits neither expansion nor delamination upon longer contact time with a composition containing an oxidizing agent.

When the multilayer foil (F) contains the three layers (P1), (P2) and (BS) described above, suitable and inventive arrangements of the layers are described below (from the interior space (in contact with the cosmetic composition (KM)) to the outside):

a) *interior space*-layer (P1)-layer (P2)-barrier layer (BS)-*outside*,
b) *interior space*-layer (P1)-barrier layer (BS)-layer (P2)-*outside*,
c) *interior space*-layer (P2)-layer (P1)-barrier layer (BS)-*outside*,
d) *interior space*-layer (P2)-barrier layer (BS)-layer (P1)-*outside*,
e) *interior space*-barrier layer (BS)-layer (P1)-layer (P2)-*outside*,
*interior space*-barrier layer (BS)-layer (P2)-layer (P1)-*outside*.

As contemplated herein, the first polymeric material of the first layer (P1) is an organic polymeric material. This material may be a layer formed from one type of polymer, or in fact a layer formed from a polymer blend. This first layer (P1) may, for example, act as the polymeric support material, i.e. when producing the foil, a layer or a film of the polymeric material (P1) may be provided and then sprayed, laminated or coated with the further layers as contemplated herein. Preferred embodiments of the present disclosure are exemplified in that the at least one first polymer layer (P1) is formed from polypropylene, polyethylene, polyester, polyamide or polyvinyl alcohol, in particular from polypropylene. In the context of the present disclosure, the term “is formed from” should be understood to mean that the polymer layer contains at least about 70% by weight, advantageously at least about 80% by weight, preferably at least about 90% by weight, in particular at least about 99% by weight of the compounds listed above, respectively with respect to the total weight of the polymer layer (P1).

A particularly preferred product as contemplated herein is thus exemplified in that the multilayer foil (F) comprises at least one first polymer layer (P1) which is formed from polypropylene. Polypropylene is alternatively also known as poly(l-methylethylene) and is a thermoplastic polymer which belongs to the polyolefins group. Polypropylene is produced by polymerization of propylene (propene) using various catalysts. Thus, for example, polypropylene can be produced by stereospecific polymerization of propylene in the gas phase or in suspension using a Giulio Natta type polymerization. Polypropylenes as contemplated herein may be isotactic and thus highly crystalline, but may also be syndiotactic or amorphous. The average relative molar mass may be controlled, for example, by setting a specific hydrogen partial pressure during polymerization of the propene. As an example, polypropylene may have average relative molar masses of approximately 150000 to about 1500000 g/mol. Polypropylene may, for example, be processed by extrusion and blow moulding, or by compression, calendering, thermoforming and cold forming.

Preferably, the first polymer layer (P1) has a specific layer thickness. Thus, in the context of the present disclosure, the at least one first polymer layer (P1) preferably has a layer thickness of from about 20.0 μm to about 300 μm, preferably of from about 40.0 μm to about 200 μm, preferably of from about 50.0 μm to about 100 μm, in particular of from about 60.0 μm to about 90.0 μm.

A particularly preferred product as contemplated herein comprises a multilayer foil (F) with at least one first polymer layer (P1) which is formed from polypropylene and has a layer thickness of from about 60.0 to about 90.0 μm.

Furthermore, the multilayer foil (F) from which the package is produced comprises a second polymer layer (P2) formed from a second polymeric material. The second polymeric material may be a layer formed from one type of polymer, or in fact a layer formed from a polymer blend. When producing the multi-layered foil, the second layer (P2)—either before or after application of the barrier layer (BS)—may, for example, be sprayed, applied or coated onto the first polymer layer (P1) which functions as the support layer. However, the second polymer layer (P2) may also be envisaged as functioning as the support layer onto which the barrier layer (BS) and the first polymer layer (P1) are applied.

As a function of the lay-up sequence described above, the first polymeric material of the first polymer layer (P1) and the second polymeric material of the second polymer layer (P2) may either be the same (insofar as both layers do not come into mutual contact) or may also be different. Thus, the polymer layer (P2) may be formed from the compounds described in connection with the polymer layer (P1). Preferably, the layers (P1) and (P2) may be prepared from different polymeric materials (i.e. different polymers or polymer blends). Thus, in the context of the present disclosure, it is preferable for the at least one second polymer layer (P2) to be formed from polyethylene terephthalate or from polyethylene naphthalate, in particular from polyethylene terephthalate. In the context of the present disclosure, the term “is formed from” should be understood to mean that the polymer layer contains at least about 70% by weight, advantageously at least about 80% by weight, preferably at least about 90% by weight, in particular at least about 99% by weight, respectively with respect to the total weight of the polymer layer (P2), of the compounds listed above. Polyethylene terephthalate (PET) is a polymer from the polyester group. Polyethylene terephthalate may, for example, be produced by transesterification of dimethylterephthalate with ethylene glycol at high temperatures. In this transesterification reaction, methanol is eliminated, which is removed by distillation. The bis(2-hydroxyethyl)terephthalate which is formed is reacted to form PET by polycondensation, whereupon ethylene glycol is again formed. A further method for the production of polyethylene terephthalate is the direct polycondensation of ethylene glycol and terephthalic acid at high temperatures and distilling off the water that is formed.

Preferably, the second polymer layer (P2) is thinner than the polymer layer (P1). Thus, in the context of the present disclosure, the at least one second polymer layer (P2) advantageously has a layer thickness of from about 1.00 μm to about 100 μm, advantageously of from about 2.50 μm to about 50.0 μm, preferably of from about 5.00 μm to about 25.0 μm, in particular of from about 10.0 μm to about 20.0 μm.

A particularly preferred product as contemplated herein is thus exemplified in that the multilayer foil (F) comprises at least one second polymer layer (P2), which is formed from polyethylene terephthalate and which has a layer thickness of from about 10.0 to about 20.0 μm.

The polymer layers (P1) and (P2) of the multilayer foil (F) include organic polymeric materials which as a rule do not have a sufficient barrier action against gases and water vapour. If the composition (KM) containing an oxidizing agent is packaged in a package (VP) formed from a multilayer foil (F) which comprises only the two organic polymer layers (P1) and (P2), then water vapour can escape unhindered, so that the water content in the composition (KM) varies in an unacceptable manner upon long-term storage. In order to specifically minimize the uncontrolled escape of water vapour from the package (VP), the organic polymer layers (P1) and (P2) are thus laminated with a barrier layer (BS).

The barrier layer (BS) acts to block the passage of gases and water vapour. In the context of the present disclosure, this means that the barrier layer (BS) reduces the permeation rate of water vapour and of gases through the foil. A foil (F) as contemplated herein which has a barrier layer (BS) in addition to the layers (P1) and (P2) thus has a reduced water vapour permeability and a reduced permeability to gas compared with a comparable foil (with the same total thickness) which has only the two layers (P1) and (P2) but no barrier layer (BS).

As an example, the barrier layer (BS) is a thin layer which comprises an inorganic material, wherein the inorganic material may be applied with the aid of vacuum coating techniques (for example PVD (physical vapour deposition) or CVD (chemical vapour deposition)) onto the organic polymer layer (P1) and/or (P2).

If the barrier layer (BS) is a layer which comprises at least one inorganic material, then, for example, the following may be considered: aluminium, aluminium oxides, magnesium, magnesium oxides, silicon, silicon oxides, titanium, titanium oxides, tin, tin oxides, zirconium, zirconium oxides and/or carbon. In this context, oxides which may be selected from the group formed by aluminium oxides, magnesium oxides, silicon oxides, titanium oxides, tin oxides and/or zirconium oxides are particularly preferred. More particularly preferably, the barrier layer (BS) formed from inorganic material is located between the two polymer layers (P1) and (P2). The production of foils with barrier layers formed from inorganic material is described, for example, in the document EP 1 036 813 A1; the full content thereof is hereby incorporated by reference.

The barrier layer (BS) may also comprise a thin layer of inorganic-organic hybrid polymers. These polymers are known in the literature by the specialist term ORMOCER polymers. A typical ORMOCER polymer can, for example, be produced by hydrolytic polycondensation of an organo-functional silane with an aluminium compound and optionally with an inorganic oxide component. Examples of relevant syntheses are disclosed in the document EP 0 792 846 B1; the full content thereof is hereby incorporated by reference. Inorganic-organic hybrid polymers (ORMOCER polymers) have both inorganic as well as organic network structures. The inorganic silicate network structure may be built up in a sol-gel process via controlled hydrolysis and condensation of alkoxysilanes. Because metal alkoxides are additionally included in the sol-gel process, the silicate network can be specifically modified. By polymerization of organo-functional groups which are introduced into the material by employing the organoalkoxysilanes, an organic network is additionally constructed. The ORMOCER polymers produced in this manner may, for example, be applied to the layers (P1) and/or (P2) using conventional application techniques (spraying, painting, etc).

Preferred embodiments of the present disclosure are thus exemplified in that the at least one barrier layer (BS) is formed from aluminium oxides, magnesium oxides, silicon oxides, titanium oxides, tin oxides, zirconium oxides, inorganic-organic hybrid polymers (ORMOCER polymers) or mixtures thereof, in particular from silicon oxides. In the context of the present disclosure, the term “is formed from” should be understood to mean that the polymer layer contains at least about 70% by weight, advantageously at least about 80% by weight, preferably at least about 90% by weight, in particular at least about 99% by weight, respectively with respect to the total weight of the barrier layer (BS), of the compounds listed above. Multilayer foils (F) as contemplated herein in which the barrier layer (BS) is formed from silicon oxides or from inorganic-organic hybrid polymers (ORMOCER polymers) are particularly preferred.

Furthermore, it is also possible for the multilayer foil (F) which constitutes the wall of the package (VP) to have a barrier layer (BS) which comprises both inorganic oxide components as well as inorganic-organic hybrid polymers (ORMOCER polymers). In addition, the barrier layer (BS) may also comprise yet another organic polymeric material which in itself has no barrier action but, for example, which increases the mechanical stability of the barrier layer, facilitates production or acts to improve the bonding of the layers (BS) and (P1) and/or (P2). Multilayer foils (F) as contemplated herein in which the barrier layer is formed from aluminium oxides, magnesium oxides, silicon oxides, titanium oxides, tin oxides, zirconium oxides as well as mixtures thereof and additionally at least one inorganic-organic hybrid polymer (ORMOCER polymers) are particularly preferred.

The thicker the barrier layer (BS), the larger or stronger will be the blocking action to gases and water vapour. The thickness of the barrier layer (BS) may thus be selected as a function of the desired blocking barrier action. The barrier layer (BS) may, for example, have a layer thickness of from about 1 to about 1000 nm (nanometer). Preferably, the barrier layer (BS) has a layer thickness of from about 5 to about 500 nm, more preferably of from about 10 to about 250 nm and particularly preferably of from about 10 to about 150 nm (nanometer). Preferred embodiments of the present disclosure are therefore exemplified in that the at least one barrier layer (BS) has a layer thickness of from about 1.00 nm to about 1000 nm, advantageously of from about 5.00 nm to about 500 nm, preferably of from about 10.0 nm to about 250 nm, in particular of from about 10.0 nm to about 150 nm.

In addition to the layers (P1), (P2) and (BS) described above, the multilayer foil (F) may also additionally comprise one or more further layers. These further layers may, for example, be intermediate layers and/or bonding layers. Thus, as contemplated herein, the at least one multilayer foil (F) preferably additionally contains at least one further layer selected from the group formed by intermediate layers (SZ), bonding layers (SK) and mixtures thereof.

By way of example, the foils (F) may have further intermediate layers (SZ) in order to increase the mechanical stability. Intermediate layers may also prevent or minimize the permeation of polymers or residual monomers from a polymer layer into the cosmetic composition (KM).

In order to increase the bond strength, the foils may additionally comprise one or more bonding layers (SK) in order to reduce or prevent delamination (i.e. detachment or the formation of an air gap) between two layers.

A particularly preferred product as contemplated herein is exemplified in that, in addition to the first polymer layer (P1), the second polymer layer (P2) and the barrier layer (BS), the multilayer foil (F) additionally contains one or more further layers which are selected from intermediate layers (SZ) and/or bonding layers (SK).

If the multilayer foil (F) contains yet more layers in addition to the layers (P1), (P2) and (BS), then the following suitable and inventive arrangements of the layers can be described (from the interior space (in contact with the cosmetic composition (KM)) to the outside):

a) *interior space*-layer (P1)-first bonding layer (SK1)-layer (P2)-second bonding layer (SK2)-barrier layer (BS)-*outside*,
b) *interior space*-layer (P1)-bonding layer (SK1)-layer (P2)-barrier layer (BS)-*outside*,
c) *interior space*-layer (P1)-layer (P2)-second bonding layer (SK2)-barrier layer (BS)-*outside*,
d) *interior space*-barrier layer (BS)-first bonding layer (SK1)-layer (P1)-second bonding layer (SK2)-layer (P2)-*outside*,
e) *interior space*-barrier layer (BS)-bonding layer (SK)-layer (P1)-layer (P2)-*outside*,
f) *interior space*-barrier layer (BS)-layer (S1)-bonding layer (SK)-layer (P2)-*outside*,
g) *interior space*-layer (P1)-first bonding layer (SK1)-barrier layer (BS)-second bonding layer (SK2)-layer (P2)-*outside*,
h) *interior space*-layer (P1)-bonding layer (SK)-barrier layer (BS)-layer (P2)-*outside*,
i) *interior space*-layer (P1)-barrier layer (BS)-bonding layer (SK)-layer (P2)-*outside*

As the second component, the product as contemplated herein comprises a cosmetic composition (KM) which is packaged in the package (VP) and comprises at least one oxidizing agent, a special thickening agent as well as an anionic surfactant.

The product as contemplated herein is intended to change colour oxidatively. To this end—as already described—a cosmetic composition (KM) which contains an oxidizing agent is usually mixed with a second preparation (B) which has been packaged separately from (KM). In this manner, the ready-to-use oxidative coloration agent is produced. The preparation (B) may contain different ingredients, depending on whether the oxidative colour change is intended to involve bleaching, lightening or colouration. When just lightening or bleaching is to be carried out, the preparation (B) contains at least one alkalizing agent. If an oxidative colouration is desired, then in addition to the alkalizing agent, the preparation (B) also often contains oxidative dye precursors. In order to ensure a sufficiently rapid miscibility of the preparations (KM) and (B), both the preparation (KM) and also the preparation (B) are usually non-viscous, aqueous or water-containing preparations.

The preparation (KM) as contemplated herein is an aqueous preparation. The water content of the preparation (KM) may, for example—with respect to the total weight of the preparation (KM)—be from about 60 to about 97% by weight, preferably from about 75 to about 93% by weight, preferably from about 78 to about 91% by weight, in particular from about 80 to about 88.0% by weight. All of the details regarding weight given as a % by weight here are with respect to the total weight of water contained in the preparation (KM) which are in relation to the total weight of the preparation (KM).

As the first essential ingredient a), the cosmetic preparation (KM) contains at least one oxidizing agent. Preferably in this regard, specific oxidizing agents are employed. Thus, in the context of the present disclosure, advantageously, the cosmetic composition (KM) contains at least one oxidizing compound selected from the group formed by persulphates, chlorites, hydrogen peroxide and addition products of hydrogen peroxide to urea, melamine as well as sodium borate, in particular hydrogen peroxide. The use of hydrogen peroxide has been shown to be particularly advantageous in the context of the present disclosure.

The concentration of the oxidizing agent in the composition (KM) is determined on the one hand by legal requirements and on the other hand on the desired effect; preferably, from about 0.5 to about 20.0% by weight solutions in water are used. Thus, as contemplated herein, the cosmetic composition (KM) preferably contains the at least one oxidizing compound, in particular hydrogen peroxide, in a total quantity of from about 0.5 to about 20% by weight, preferably of from about 1.0 to about 18% by weight, preferably of from about 1.2 to about 16% by weight, in particular of from about 1.5 to about 15% by weight, with respect to the total weight of the cosmetic composition (KM). The higher the oxidizing agent content, in particular of hydrogen peroxide, in the composition (KM), the greater is the quantity of gas arising during partial decomposition of the oxidizing agent. Preparations containing higher concentrations of oxidizing agent which are stable upon storage in a package (VP) are therefore much more difficult to package than preparations with a lower concentration. The quantity of hydrogen peroxide here is with respect to 100% hydrogen peroxide.

During the course of the work carried out in respect of this present disclosure, it was shown that the product as contemplated herein is also particularly suitable for the packaging and stable storage of more concentrated hydrogen peroxide preparations (KM). Thus, packages (VP) which contain preparations (KM) containing from about 9 to about 12% by weight of hydrogen peroxide did not exhibit any variation in volume (i.e. no expansion) and no accidental opening (i.e. the packages did not burst) even after storage at high temperatures for several weeks.

The cosmetic preparation (KM) contains at least one special thickening agent as the second essential ingredient b). In this connection, copolymers of (meth)acrylic acid and acrylic acid ethyl esters have been shown to be particularly advantageous, because even under highly acidic conditions and in the presence of an oxidizing agent, these copolymers can guarantee sufficient thickening over a long period of time. The thickening ensures that the cosmetic composition (KM) is easy to handle. In order to ensure sufficient thickening, the at least one thickening agent is preferably used in specific ranges of quantities. Thus, in the context of the present disclosure, advantageously, the cosmetic preparation (KM) contains the at least one thickening agent, in particular copolymers of (meth)acrylic acid and acrylic acid ethyl esters, in a total quantity of from about 0.20 to about 6.0% by weight, preferably of from about 0.25 to about 5.5% by weight, preferably of from about 0.30 to about 5.0% by weight, in particular of from about 0.40 to about 4.5% by weight, with respect to the total weight of the cosmetic composition (KM). In the context of the present disclosure, copolymers which are commercially available under the trade name Aculyn 33A have been shown to be particularly advantageous.

The cosmetic composition (KM) contains at least one anionic surfactant as the third essential ingredient. The use of these surfactants ensures sufficient miscibility of the cosmetic agent (KM) with the preparation (B) which contains the oxidative dye precursor and also ensures good stability upon storage, because precipitation of components of the cosmetic composition (KM) is prevented. Preferred embodiments of the present disclosure are therefore exemplified in that the cosmetic composition (KM) contains at least one anionic surfactant selected from the group formed by (i) C₈-C₁₈ alkylether sulphates with from about 1 to about 10 mol of ethylene oxide per mol of C₈-C₁₈ alkylether sulphate as well as their salts, in particular sodium salts of C₁₂-C₁₄ alkylether sulphates with about 2 mol of ethylene oxide per mol of C₁₂-C₁₄ alkylether sulphate, (ii) C₈-C₁₈ alkyl sulphates, (iii) salts of linear and branched C₈-C₃₀ carboxylic acids, (iv) acyl sarcosides containing from about 8 to about 24 carbon atoms in the acyl group, (v) acyl taurides containing from about 8 to about 24 carbon atoms in the acyl group, (vi) acyl isethionates containing from about 8 to about 24 carbon atoms in the acyl group, (vii) sulphosuccinic acid mono- and dialkyl esters containing from about 8 to about 24 carbon atoms in the alkyl group as well as sulphosuccinic acid monoalkyl polyoxyethyl esters containing from about 8 to about 24 carbon atoms in the alkyl group and from about 1 to about 6, preferably from about 1 to about 4 oxyethyl groups, (viii) linear alpha-olefin sulphonates containing from about 8 to about 24 carbon atoms, (ix) alpha-sulphocarboxylic acid methyl esters of carboxylic acids containing from about 8 to about 30 carbon atoms, (x) alkyl- and/or alkenyl ether phosphates, (xi) sulphonates of unsaturated carboxylic acids containing from about 8 to about 24 carbon atoms and from about 1 to about 6 double bonds, (xii) C₈-C₃₀ ethercarboxylates with from about 1 to about 10 mol of ethylene oxide per mol of C₈-C₃₀ ethercarboxylate, as well as (xiii) mixtures thereof, in particular sodium salts of C₁₂-C₁₄ alkylether sulphates with about 2 mol of ethylene oxide per mol of C₁₂-C₁₄ alkylether sulphate.

In order to ensure sufficient dispersion of all of the ingredients of the cosmetic agent (KM), the at least one anionic surfactant is preferably used in specific total quantities. Thus, in the context of the present disclosure, preferably, the cosmetic composition (KM) contains the at least one anionic surfactant, in particular sodium salts of C₁₂-C₁₄ alkylether sulphate with about 2 mol of ethylene oxide per mol of C₁₂-C₁₄ alkylether sulphate, in a total quantity of from about 0.025 to about 2.0% by weight, preferably of from about 0.10 to about 1.5% by weight, preferably of from about 0.15 to about 1.2% by weight, in particular of from about 0.27 to about 1.0% by weight, with respect to the total weight of the cosmetic composition (KM).

During the course of the studies leading to this present disclosure, it was shown that the use of a special thickening agent as mentioned above as well as an anionic surfactant ensures that the cosmetic composition (KM) which contains at least one oxidizing agent can be packaged and stored in the special package (VP) without this package—which has a barrier layer with a blocking action for gases and water vapour—expanding or bursting.

In this connection, a quite specific combination of oxidizing agent, thickening agent and anionic surfactant has been shown to be advantageous. In a preferred embodiment, then, the product as contemplated herein is exemplified in that the cosmetic composition (KM) contains hydrogen peroxide, a copolymer of (meth)acrylic acid and acrylic acid ethyl ester as well as sodium salts of C₁₂-C₁₄ alkylether sulphate with about 2 mol of ethylene oxide per mol of C₁₂-C₁₄ alkylether sulphate.

In order to further optimize the stability upon storage, the quantities of the aforementioned compounds in the preparation (KM) are preferably employed in specific ranges. Particularly preferred embodiments are thus exemplified in that the cosmetic composition (KM) contains from about 1.5 to about 15% by weight of hydrogen peroxide, from about 1.5 to about 15% by weight of a copolymer of (meth)acrylic acid and acrylic acid ethyl ester, as well as from about 0.27 to about 1.0% by weight of a sodium salt of C₁₂-C₁₄ alkylether sulphates with about 2 mol of ethylene oxide per mol of C₁₂-C₁₄ alkylether sulphate, respectively with respect to the total weight of the cosmetic agent.

The cosmetic composition (KM) preferably has an acidic pH in order to prevent or reduce decomposition of the oxidizing agent used, in particular of hydrogen peroxide. Thus, in the context of the present disclosure, preferably, the cosmetic composition (KM) has a pH (measured at 20° C.) of from about pH 1.5 to about pH 5.0, advantageously of from about pH 2.0 to about pH 4.6, preferably of from about pH 2.3 to about pH 4.5, in particular of from about pH 2.5 to about pH 4.0.

The preparation (KM) in the package (VP) contains the essential ingredients in an aqueous or hydro-alcoholic carrier which may, for example, be a cream, an emulsion, a gel or in fact a surfactant-containing foaming solution. In order to provide these forms of administration with the desired properties, the preparation (KM) may also contain additional active substances, auxiliary substances and additives.

The preparation (KM) may, for example, additionally contain another or several more acids in order to stabilize the oxidizing agent used, in particular hydrogen peroxide. Thus, in the context of the present disclosure, preferably, the cosmetic composition (KM) additionally contains at least one acid selected from the group formed by dipicolinic acid, citric acid, acetic acid, malic acid, lactic acid, tartaric acid, hydrochloric acid, phosphoric acid, pyrophosphoric acid and salts thereof, benzoic acid as well as its salts, 1-hydroxyethane-1,1-diphosphonic acid, ethylene diamine tetraacetic acid and salts thereof, sulphuric acid as well as mixtures, in particular a mixture of dipicolinic acid, disodium pyrophosphate, ethylene diamine tetraacetic acid as well as salts thereof and 1-hydroxyethane-1,1-diphosphonic acid.

Particularly good stabilization of the oxidizing agent, in particular of hydrogen peroxide, is obtained when the quantities of the acids mentioned above are used in specific ranges. Thus, in this context, advantageously, the at least one acid, in particular the mixture of dipicolinic acid, disodium pyrophosphate, ethylenediamine tetraacetic acid as well as salts thereof and 1-hydroxyethane-1,1-diphosphonic acid, is used in a total quantity of from about 0.1 to about 3.0% by weight, advantageously of from about 0.5 to about 2.5% by weight, preferably of from about 0.8 to about 2.0% by weight, in particular of from about 0.9 to about 1.5% by weight, with respect to the total weight of the cosmetic composition (KM).

The following tables show particularly preferred embodiments AF 1 to AF 12 of the cosmetic composition (KM) contained in the package (VP) (all details as a % by weight unless indicated otherwise)

| | AF 1 | AF 2 | AF 3 | AF 4 |
|---|---|---|---|---|
| Oxidizing agent | 0.5-20 | 1.0-18 | 1.2-16 | 1.5-15 |
| Thickening agent ²⁾ | 0.20-6.0 | 0.25-5.5 | 0.30-5.0 | 0.40-4.5 |
| Anionic surfactant | 0.025-2.0 | 0.10-1.5 | 0.15-1.2 | 0.27-1.0 |
| Cosmetic support ¹⁾ | ad 100 | ad 100 | ad 100 | ad 100 |

| | AF 5 | AF 6 | AF 7 | AF 8 |
|---|---|---|---|---|
| Oxidizing agent ³⁾ | 0.5-20 | 1.0-18 | 1.2-16 | 1.5-15 |
| Thickening agent ²⁾ | 0.20-6.0 | 0.25-5.5 | 0.30-5.0 | 0.40-4.5 |
| Anionic surfactant ⁴⁾ | 0.025-2.0 | 0.10-1.5 | 0.15-1.2 | 0.27-1.0 |
| Cosmetic support ¹⁾ | ad 100 | ad 100 | ad 100 | ad 100 |

| | AF 9 | AF 10 | AF 11 | AF 12 |
|---|---|---|---|---|
| Oxidizing agent ³⁾ | 0.5-20 | 1.0-18 | 1.2-16 | 1.5-15 |
| Thickening agent ⁵⁾ | 0.20-6.0 | 0.25-5.5 | 0.30-5.0 | 0.40-4.5 |
| Anionic surfactant ⁶⁾ | 0.025-2.0 | 0.10-1.5 | 0.15-1.2 | 0.27-1.0 |
| Cosmetic support ¹⁾ | ad 100 | ad 100 | ad 100 | ad 100 |

¹⁾ aqueous or hydro-alcoholic support,
²⁾ selected from the group formed by copolymers of (meth)acrylic acid and (meth)acrylic acid esters, copolymers of (meth)acrylates and (meth)acrylamides, copolymers of hydroxyethyl(meth)acrylates and (meth)acrylamides, copolymers of (meth)acrylates, (meth)acrylamides and ethoxylated (meth)acrylic acid esters as well as mixtures thereof,
³⁾ hydrogen peroxide, quantity with respect to 100% hydrogen peroxide,
⁴⁾ selected from the group formed by C₈-C₁₈ alkylether sulphates with 1 to 10 mol of ethylene oxide per mol of C₈-C₁₈ alkylether sulphate as well as their salts,
⁵⁾ selected from copolymers of (meth)acrylic acid and acrylic acid ethyl esters,
⁶⁾ selected from sodium salts of C₁₂-C₁₄ alkylether sulphates with 2 mol of ethylene oxide per mol of C₁₂-C₁₄ alkylether sulphate.

The embodiments AF 1 to 12 described above were packaged into respective packages (VP) which had the following arrangement of the multilayer foil (F) (from the interior space (in contact with the cosmetic composition (KM)) to the outside):

a) *interior space*-layer (P1)-layer (P2)-barrier layer (BS)-*outside*,
b) *interior space*-layer (P1)-barrier layer (BS)-layer (P2)-*outside*,
c) *interior space*-layer (P2)-layer (P1)-barrier layer (BS)-*outside*,
d) *interior space*-layer (P2)-barrier layer (BS)-layer (P1)-*outside*,
e) *interior space*-barrier layer (BS)-layer (P1)-layer (P2)-*outside*,
f) *interior space*-barrier layer (BS)-layer (P2)-layer (P1)-*outside*,
g) *interior space*-layer (P1)-first bonding layer (SK1)-layer (P2)-second bonding layer (SK2)-barrier layer (BS)-*outside*,
h) *interior space*-layer (P1)-bonding layer (SK1)-layer (P2)-barrier layer (BS)-*outside*,
i) *interior space*-layer (P1)-layer (P2)-second bonding layer (SK2)-barrier layer (BS)-*outside*,
j) *interior space*-barrier layer (BS)-first bonding layer (SK1)-layer (P1)-second bonding layer (SK2)-layer (P2)-*outside*,
k) *interior space*-barrier layer (BS)-bonding layer (SK)-layer (P1)-layer (P2)-*outside*,
l) *interior space*-barrier layer (BS)-layer (S1)-bonding layer (SK)-layer (P2)-*outside*,
m) *interior space*-layer (P1)-first bonding layer (SK1)-barrier layer (BS)-second bonding layer (SK2)-layer (P2)-*outside*,
n) *interior space*-layer (P1)-bonding layer (SK)-barrier layer (BS)-layer (P2)-*outside*,
o) *interior space*-layer (P1)-barrier layer (BS)-bonding layer (SK)-layer (P2)-*outside*.

The products as contemplated herein that can be obtained in this manner have a high stability on storage as well as a water loss during storage which is in an acceptable range. No expansion or delamination of the package (VP) was observed during storage of these cosmetic products as contemplated herein.

The product as contemplated herein is used for the purposes of oxidative coloration. To this end, the preparation (KM) packaged in the package (VP) which comprises the oxidizing agent preparation is mixed with at least one further preparation (B) in order to produce the ready-to-use coloration agent. In order to avoid incompatibilities or to avoid a premature reaction, the preparations (KM) and (B) are packaged separately from each other.

A particularly preferred product as contemplated herein is exemplified in that it comprises a preparation (B) which is packaged separately from the preparation (KM), wherein the preparation (B) contains at least one compound selected from oxidative dye precursors, direct dyes, alkalizing agents as well as mixtures thereof. Preferred products of the present disclosure are thus exemplified in that they additionally comprise at least one second cosmetic composition (KM2) which contains at least one compound selected from oxidative dye precursors, direct dyes, alkalizing agents as well as mixtures thereof and which is packaged separately from the cosmetic composition (KM).

If oxidative coloration is desired, the preparation (B) contains at least one oxidative dye precursor. Oxidative dye precursors can be divided into developers and couplers, wherein the developers, because of their greater sensitivity as regards oxygen, are usually used in the form of their physiologically acceptable salts (for example in the form of their hydrochlorides, hydrobromides, hydrogen sulphates or sulphates). In the context of oxidative colouring, coupler components alone do not bring about any significant coloration, but always require the presence of developer components. Preferably, agents of this type contain at least one oxidative dye precursor of the developer type and at least one oxidative dye precursor of the coupler type. Particularly suitable oxidative dye precursors of the developer type are thus selected from at least one compound from the group formed by p-phenylenediamine, p-toluylenediamine, 2-(2-hydroxyethyl)-p-phenylenediamine, 2-(1,2-dihydroxyethyl)-p-phenylenediamine, N,N-bis-(2-hydroxyethyl)-p-phenylenediamine, 2-methoxymethyl-p-phenylenediamine, N-(4-amino-3-methylphenyl)-N-[3-(1H-imidazol-1-yl)propyl]amine, N,N′-bis-(2-hydroxyethyl)-N,N′-bis-(4-aminophenyl)-1,3-diamino-propan-2-ol, bis-(2-hydroxy-5-aminophenyl)methane, 1,3-bis-(2,5-diaminophenoxy)propan-2-ol, N,N′-bis-(4-aminophenyl)-1,4-diazacycloheptane, 1,10-bis-(2,5-diaminophenyl)-1,4,7,10-tetraoxadecane, p-aminophenol, 4-amino-3-methylphenol, 4-amino-2-aminomethylphenol, 4-amino-2-(1,2-dihydroxyethyl)phenol, 4-amino-2-(diethylaminomethyl)phenol, 4,5-diamino-1-(2-hydroxyethyl)pyrazole, 2,4,5,6-tetraaminopyrimidine, 4-hydroxy-2,5,6-triaminopyrimidine, 2-hydroxy-4,5,6-triaminopyrimidine, 2,3-diamino-6,7-dihydro-1H,5H-pyrazolo[1,2-a]pyrazol-1-one, as well as their physiologically acceptable salts.

Particularly preferred oxidative dye precursors of the coupler type are thus selected from the group formed by 3-aminophenol, 5-amino-2-methylphenol, 3-amino-2-chloro-6-methylphenol, 2-hydroxy-4-aminophenoxyethanol, 5-amino-4-chloro-2-methylphenol, 5-(2-hydroxyethyl)-amino-2-methylphenol, 2,4-dichloro-3-aminophenol, 2-aminophenol, 3-phenylenediamine, 2-(2,4-diaminophenoxy)ethanol, 1,3-bis(2,4-diaminophenoxy)propane, 1-methoxy-2-amino-4-(2-hydroxyethylamino)benzene, 1,3-bis(2,4-diaminophenyl)propane, 2,6-bis(2′-hydroxyethylamino)-1-methylbenzene, 2-({3-[(2-hydroxyethyl)amino]-4-methoxy-5-methylphenyl}amino)ethanol, 2-({3-[(2-hydroxyethyl)amino]-2-methoxy-5-methylphenyl}amino)ethanol, 2-({3-[(2-hydroxyethyl)amino]-4,5-dimethylphenyl}amino)ethanol, 2-[3-morpholino-4-ylphenyl)amino]ethanol, 3-amino-4-(2-methoxyethoxy)-5-methylphenylamine, 1-amino-3-bis-(2-hydroxyethyl)aminobenzene, resorcinol, 2-methylresorcinol, 4-chlororesorcinol, 1,2,4-trihydroxybenzene, 2-amino-3-hydroxypyridine, 3-amino-2-methylamino-6-methoxypyridine, 2,6-dihydroxy-3,4-dimethylpyridine, 3,5-diamino-2,6-dimethoxypyridine, 1-phenyl-3-methylpyrazol-5-one, 1-naphthol, 1,5-dihydroxynaphthalene, 2,7-dihydroxynaphthalene, 1,7-dihydroxynaphthalene, 1,8-dihydroxynaphthalene, 4-hydroxyindole, 6-hydroxyindole, 7-hydroxyindole, 4-hydroxyindoline, 6-hydroxyindoline, 7-hydroxyindoline or mixtures of these compounds, or their physiologically acceptable salts.

In addition, the preparation (B) may also contain one or more direct dyes. Suitable non-ionic direct dyes may be selected from the group formed by HC Yellow 2, HC Yellow 4, HC Yellow 5, HC Yellow 6, HC Yellow 12, HC Orange 1, Disperse Orange 3, HC Red 1, HC Red 3, HC Red 7, HC Red 10, HC Red 11, HC Red 13, HC Red BN, HC Blue 2, HC Blue 11, HC Blue 12, Disperse Blue 3, HC Violet 1, Disperse Violet 1, Disperse Violet 4, Disperse Black 9, 1,4-diamino-2-nitrobenzene, 2-amino-4-nitrophenol, 1,4-bis-(2-hydroxyethyl)-amino-2-nitrobenzene, 3-nitro-4-(2-hydroxyethyl)aminophenol, 2-(2-hydroxyethyl)amino-4,6-dinitrophenol, 4-[(2-hydroxyethyl)amino]-3-nitro-1-methylbenzene, 1-amino-4-(2-hydroxyethyl)amino-5-chloro-2-nitrobenzene, 4-amino-3-nitrophenol, 1-(2′-ureidoethyl)amino-4-nitrobenzene, 2-[(4-amino-2-nitrophenyl)amino]benzoic acid, 4-[(3-hydroxypropyl)amino]-3-nitrophenol, 4-nitro-o-phenylenediamine, 6-nitro-1,2,3,4-tetrahydroquinoxaline, 2-hydroxy-1,4-naphthoquinone, picramic acid and their salts, 2-amino-6-chloro-4-nitrophenol, 4-ethylamino-3-nitrobenzoic acid and 2-chloro-6-ethylamino-4-nitrophenol.

Suitable anionic direct dyes may be selected from the group formed by Acid Yellow 1, Yellow 10, Acid Yellow 23, Acid Yellow 36, Acid Orange 7, Acid Red 33, Acid Red 52, Pigment Red 57:1, Acid Blue 7, Acid Green 50, Acid Violet 43, Acid Black 1, Acid Black 52, bromophenol blue and tetrabromophenol blue.

Suitable cationic direct dyes are cationic triphenylmethane dyes such as, for example, Basic Blue 7, Basic Blue 26, Basic Violet 2 and Basic Violet 14, aromatic systems which have been substituted with a quaternary nitrogen group such as, for example, Basic Yellow 57, Basic Red 76, Basic Blue 99, Basic Brown 16 and Basic Brown 17, cationic anthraquinone dyes such as HC Blue 16 (Bluequat (B)), as well as direct dyes which contain a heterocycle containing at least one quaternary nitrogen atom, in particular Basic Yellow 87, Basic Orange 31 and Basic Red 51. The cationic direct dyes which are marketed under the trade name Arianor are also cationic direct dyes which are suitable for use in the present disclosure.

Colouring processes on keratin fibres are usually carried out in an alkaline medium. In order to care for the keratin fibres and also the skin as much as possible, however, setting the pH too high is not desirable. Thus, preferably, the pH of the medium (B) is between from about 7 and about 11, in particular between from about 8 and about 10.5. The pHs in the context of the present disclosure are pH values which are measured at a temperature of 22° C.

The preparation (B) may contain at least one alkalizing agent. The alkalizing agents which are used as contemplated herein to set the preferred pH may be selected from the group formed by ammonia, alkanolamines, basic amino acids, as well as inorganic alkalizing agents such as alkali (alkaline earth) metal hydroxides, alkali (alkaline earth) metal metasilicates, alkali (alkaline earth) metal phosphates and alkali (alkaline earth) metal hydrogen phosphates. Preferred inorganic alkalizing agents are magnesium carbonate, sodium hydroxide, potassium hydroxide, sodium silicate and sodium metasilicate. Organic alkalizing agents which may be used as contemplated herein are preferably selected from monoethanolamine, 2-amino-2-methylpropanol and triethanolamine. The basic amino acids which may be used as the alkalizing agent as contemplated herein are preferably selected from the group formed by arginine, lysine, ornithine and histidine, particularly preferably arginine. However, in the context of tests carried out in respect of the present disclosure, it has been shown that further preferred agents for the present disclosure are exemplified in that they additionally contain an organic alkalizing agent. One embodiment of the first aspect of the present disclosure is exemplified in that the agent additionally contains at least one alkalizing agent which is selected from the group formed by ammonia, alkanolamines and basic amino acids, in particular from ammonia, monoethanolamine and arginine or its acceptable salts.

The preparation (B) may furthermore contain additional active substances, auxiliary substances and additives. Thus, for example, it may contain one or more fats from the group formed by C₁₂-C₃₀ fatty alcohols, C₁₂-C₃₀ fatty acid triglycerides, C₁₂-C₃₀ fatty acid monoglycerides, C₁₂-C₃₀ fatty acid diglycerides and/or hydrocarbons.

Preferably, preparation (B) may additionally contain a surface-active substance wherein, depending on the field of application, such surface-active substances are described as surfactants or as emulsifying agents: they are preferably selected from anionic, zwitterionic, amphoteric and non-ionic surfactants and emulsifying agents.

Preferably, the preparation (B) contains at least one anionic surfactant. Preferred anionic surfactants are fatty acids, alkyl sulphates, alkylether sulphates and ether carboxylic acids containing from about 10 to about 20 C atoms in the alkyl group and up to about 16 glycol ether groups in the molecule.

Furthermore, the preparation (B) may additionally contain at least one zwitterionic surfactant. Preferred zwitterionic surfactants are betaines, N-alkyl-N,N-dimethylammonium glycinates, N-acyl-aminopropyl-N,N-dimethylammonium glycinates, and 2-alkyl-3-carboxymethyl-3-hydroxyethyl imidazolines. A preferred zwitterionic surfactant is known by the INCI name cocamidopropyl betaine.

Furthermore, the preparation (B) may contain at least one amphoteric surfactant. Preferred amphoteric surfactants are N-alkylglycines, N-alkylpropionic acids, N-alkylaminobutyric acids, N-alkyliminodipropionic acids, N-hydroxyethyl-N-alkylamidopropylglycines, N-alkyltaurines, N-alkylsarcosines, 2-alkylaminopropionic acids and alkylaminoacetic acids. Particularly preferred amphoteric surfactants are N-cocoalkylaminopropionate, as cocoacylaminoethylaminopropionate, and C₁₂-C₁₈-acylsarcosine.

It has also been shown to be advantageous for the preparation (B) to contain further nonionogenic surface-active substances. Preferred non-ionic surfactants are alkylpolyglycosides as well as alkylene oxide addition products with fatty alcohols and fatty acids respectively with from about 2 to about 30 mol of ethylene oxide per mol of fatty alcohol or fatty acid. Preparations with excellent properties are also obtained when they contain fatty acid esters of ethoxylated glycerine as the non-ionic surfactants.

The non-ionic, zwitterionic or amphoteric surfactants are used in proportions of from about 0.1 to about 45% by weight, preferably from about 1 to about 30% by weight, and more particularly preferably from about 1 to about 15% by weight with respect to the total weight of the preparation (B).

The preparation (B) may additionally contain at least one thickening agent. There are no particular restrictions that can be applied to this thickening agent. Both organic and also purely inorganic thickening agents may be used. Suitable thickening agents are anionic, synthetic polymers, cationic, synthetic polymers, naturally occurring thickening agents such as non-ionic guar gums, scleroglucan gums or xanthan gums, gum Arabic, ghatti gum, karaya gum, tragacanth gum, carrageenan gum, agar-agar, carob bean gum, pectins, alginates, starch fractions and derivatives such as amylose, amylopectin and dextrins, as well as cellulose derivatives such as, for example, methylcellulose, carboxyalkylcelluloses and hydroxyalkylcelluloses, non-ionic, fully synthetic polymers such as polyvinyl alcohol or polyvinylpyrrolidinone; as well as inorganic thickening agents, in particular phyllosilicates such as, for example, bentonite, in particular smectites such as montmorillonite or hectorite.

Furthermore, the preparation (B) may contain other active substances, auxiliary substances and additives such as, for example, non-ionic polymers such as, for example, vinylpyrrolidinone/vinylacrylate copolymers, polyvinylpyrrolidinone, vinylpyrrolidinone/vinylacetate copolymers, polyethyleneglycols and polysiloxanes; additional silicones such as volatile or non-volatile, straight chain, branched or cyclic, crosslinked or non-crosslinked polyalkylsiloxanes (such as dimethicones or cyclomethicones), polyarylsiloxanes and/or polyalkylarylsiloxanes, in particular polysiloxanes containing organofunctional groups such as substituted or unsubstituted amines (amodimethicones), carboxyl-, alkoxy- and/or hydroxyl groups (dimethicone copolyols), linear polysiloxane(A)-polyoxyalkylene(B) block copolymers, grafted silicone polymers; cationic polymers such as quaternized cellulose ethers, polysiloxanes containing quaternary groups, dimethyldiallylammonium chloride polymers, acrylamide-dimethyldiallyl ammonium chloride copolymers, dimethylaminoethylmethacrylate-vinylpyrrolidinone copolymers quaternized with diethylsulphate, vinylpyrrolidinone—imidazolinium—methochloride copolymers and quaternized polyvinyl alcohol; zwitterionic and amphoteric polymers; anionic polymers such as, for example, polyacrylic acids or crosslinked polyacrylic acids; structuring agents such as glucose, maleic acid and lactic acid, hair conditioning compounds such as phospholipids, for example lecithin and cephalins; fragrancing oils, dimethylisosorbide and cyclodextrins; substances that improve the structure of fibres, in particular mono-, di- and oligosaccharides such as, for example, glucose, galactose, fructose, fruit sugar and lactose; colorants to colour the agent; antidandruff substances such as piroctone olamine, zinc omadine and climbazole; amino acids and oligopeptides; animal and/or plant-based protein hydrolysates, as well as in the form of their fatty acid condensation products or optionally anionically or cationically modified derivatives; fats and plant oils; light stabilizers and UV blockers; active substances such as panthenol, pantothenic acid, pantolactone, allantoin, pyrrolidinone carboxylic acids and their salts as well as bisabolol; polyphenols, in particular hydroxycinnamic acids, 6,7-dihydroxycoumarins, hydroxybenzoic acids, catechins, tannins, leucoanthocyanidins, anthocyanidins, flavanones, flavones and flavonols; ceramides or pseudoceramides; vitamins, provitamins and vitamin precursors; plant extracts; fats and waxes such as fatty alcohols, beeswax, montan wax and paraffins; swelling and penetrating agents such as glycerine, propylene glycol monoethyl ether, carbonates, hydrogen carbonates, guanidines, ureas as well as primary, secondary and tertiary phosphates; opacifiers such as latex, styrene/PVP and styrene/acrylamide copolymers; pearlescent agents such as ethylene glycol mono- and distearate, as well as PEG-3-distearate, and also pigments.

The selection of these further substances will be within the purview of the person skilled in the art bearing in mind the desired properties of the preparation (B) as well as of the product as contemplated herein. Further optional components and also the quantities of those components are expressly obtainable from handbooks available to the person skilled in the art. The additional active and auxiliary substances are preferably used in preparation (B) in respective quantities of from about 0.0001 to about 25% by weight, in particular of from about 0.0005 to about 15% by weight, respectively with respect to the total weight of the preparation (B).

The following examples serve to illustrate the present disclosure without in any way limiting its scope:

### EXAMPLES

A 100 nm thick layer of silicon dioxide SiOx was deposited on a layer of foil formed from polyethylene terephthalate with a thickness of 12 μm (micrometer). Next, the SiOx layer was overlaid with approximately 3 g/m² of ORMOCER polymer and cured. A 70 μm (micrometer) layer of polypropylene was then applied to the ORMOCER layer. A package (VP) was produced from the foil.

The following cosmetic compositions (KM) were used (all details as a % by weight). The preferred anionic surfactant was a sodium salt of a C₁₂-C₁₄ alkylether sulphate with 2 mol of ethylene oxide per mol of C₁₂-C₁₄ alkylether sulphate. The preferred oxidizing agent was hydrogen peroxide. The preferred thickening agent was a copolymer of (meth)acrylic acid and acrylic acid ethyl ester.

| Ingredient | KM1 | KM2 |
|---|---|---|
| Sodium hydroxide (50%) | 0.66 | 0.66 |
| Dipicolinic acid | 0.10 | 0.10 |
| Disodium pyrophosphate | 0.030 | 0.030 |
| 1-hydroxyethane-1,1-diphosphonic acid | 1.5 | 1.5 |
| (60%) | | |
| Anionic surfactant | 0.54 | 0.54 |
| Thickening agent | 0.70 | 4.2 |
| Oxidizing agent | 12 | 12 |
| Water | ad 100 | ad 100 |

The cosmetic compositions KM1 and KM2 were respectively packed into the packages (VP) described above. Next, the packages were stored for 24 weeks at 40° C. The packages had neither expanded, nor had they delaminated.

While at least one exemplary embodiment has been presented in the foregoing detailed description, it should be appreciated that a vast number of variations exist. It should also be appreciated that the exemplary embodiment or exemplary embodiments are only examples, and are not intended to limit the scope, applicability, or configuration of the various embodiments in any way. Rather, the foregoing detailed description will provide those skilled in the art with a convenient road map for implementing an exemplary embodiment as contemplated herein. It being understood that various changes may be made in the function and arrangement of elements described in an exemplary embodiment without departing from the scope of the various embodiments as set forth in the appended claims.

1. A cosmetic product for changing the natural colour of keratinous fibres, in particular human hair, comprising:
(i) a package, comprising a multilayer foil, which comprises a first polymer layer, a second polymer layer, and a barrier layer, and
(ii) a cosmetic composition, which is packaged in the package and comprises:
a) an oxidizing compound,
b) a thickening agent selected from the group of copolymers of (meth)acrylic acid and (meth)acrylic acid esters, copolymers of (meth)acrylates and (meth)acrylamides, copolymers of hydroxyethyl(meth)acrylates and (meth)acrylamides, copolymers of (meth)acrylates, (meth)acrylamides and ethoxylated (meth)acrylic acid esters as well as mixtures thereof, and
c) an anionic surfactant.

2. The cosmetic product as claimed in claim 1, wherein the multilayer foil comprises the barrier layer disposed between the first polymer layer and the second polymer layer.

3. The cosmetic product as claimed in claim 1, wherein the first polymer layer comprises polypropylene, polyethylene, polyester, polyamide or polyvinyl alcohol.

4. The cosmetic product as claimed in claim 1, wherein the first polymer layer has a layer thickness of from about 20.0 to about 300 μm.

5. The cosmetic product as claimed in claim 1, wherein the second polymer layer comprises polyethylene terephthalate or polyethylene naphthalate.

6. The cosmetic product as claimed in claim 1, wherein the second polymer layer has a layer thickness of from about 1.00 μm to about 100 μm.

7. The cosmetic product as claimed in claim 1, wherein the barrier layer comprises aluminium oxides, magnesium oxides, silicon oxides, titanium oxides, tin oxides, zirconium oxides, inorganic-organic hybrid polymers or mixtures thereof.

8. The cosmetic product as claimed in claim 1, wherein the barrier layer has a layer thickness of from about 1.00 nm to about 1000 nm.

9. The cosmetic product as claimed in claim 1, wherein the cosmetic composition comprises the oxidizing compound in a total quantity of from about 0.5 to about 20% by weight.

10. The cosmetic product as claimed in claim 1, wherein the cosmetic composition comprises the thickening agent in a total quantity of from about 0.20 to about 6.0% by weight.

11. The cosmetic product as claimed in claim 1, wherein the thickening agent comprises a copolymer of (meth)acrylic acid and acrylic acid ethyl ester and is present in an amount of from about 0.40 to about 4.5% by weight, with respect to the total weight of the cosmetic composition.

12. The cosmetic product as claimed in claim 1, wherein the oxidizing compound comprises hydrogen peroxide and is present in a total quantity of from about 1.5 to about 15% by weight, with respect to the total weight of the cosmetic composition.

13. The cosmetic product as claimed in claim 1, wherein the anionic surfactant comprises a sodium salt of C₁₂-C₁₄ alkylether sulphate with about 2 mol of ethylene oxide per mol of C₁₂-C₁₄ alkylether sulphate, wherein the anionic surfactant is present in a total quantity of from about 0.27 to about 1.0% by weight, with respect to the total weight of the cosmetic composition.

14. The cosmetic product as claimed in claim 1, wherein:
the thickening agent comprises a copolymer of (meth)acrylic acid and acrylic acid ethyl ester and is present in an amount of from about 0.40 to about 4.5% by weight, with respect to the total weight of the cosmetic composition;
the oxidizing compound comprises hydrogen peroxide and is present in a total quantity of from about 1.5 to about 15% by weight, with respect to the total weight of the cosmetic composition;
the anionic surfactant comprises a sodium salt of C₁₂-C₁₄ alkylether sulphate with about 2 mol of ethylene oxide per mol of C₁₂-C₁₄ alkylether sulphate, wherein the anionic surfactant is present in a total quantity of from about 0.27 to about 1.0% by weight, with respect to the total weight of the cosmetic composition;
wherein the cosmetic composition further comprises water—with respect to the total weight of the cosmetic composition—in a quantity of from about 60 to about 97% by weight.

15. The cosmetic product as claimed in claim 14, wherein the cosmetic composition further comprises:
an inorganic alkalizing agent; and
a mixture of acids comprising:
at least one of dipicolinic acid, disodium pyrophosphate, ethylenediamine tetraacetic acid, or salts thereof; and
1-hydroxyethane-1,1-diphosphonic acid;
wherein the acids are present in a total quantity of from about 0.9 to about 1.5% by weight, with respect to the total weight of the cosmetic composition.

16. The cosmetic product as claimed in claim 1, wherein:
the first polymer layer comprises polypropylene and has a layer thickness of from about 60.0 μm to about 90.0 μm; and
the second polymer layer comprises polyethylene terephthalate and has a layer thickness of from about 10.0 μm to about 20.0 μm.

17. The cosmetic product as claimed in claim 1, wherein the barrier layer is a multilayer foil comprising a layer comprising silicon dioxide and a layer comprising an inorganic-organic hybrid polymer.

18. The cosmetic product as claimed in claim 1, The cosmetic product as claimed in claim 1, wherein:
the first polymer layer comprises polypropylene and has a layer thickness of from about 60.0 μm to about 90.0 μm;
the second polymer layer comprises polyethylene terephthalate and has a layer thickness of from about 10.0 μm to about 20.0 μm; and
the barrier layer comprises:
a layer comprising silicon dioxide; and
a layer comprising an inorganic-organic hybrid polymer;
wherein the barrier layer has a layer thickness of from about 10.0 nm to 150 nm and is disposed between the first polymer layer and the second polymer layer.

19. A cosmetic product for changing the natural colour of keratinous fibres comprising:
(i) a package, comprising a multilayer foil, which comprises:
a first polymer layer comprising polypropylene and having a layer thickness of from about 60.0 μm to about 90.0 μm,
a second polymer layer comprising polyethylene terephthalate and having a layer thickness of from about 10.0 μm to about 20.0 μm, and
a barrier layer comprising:
a layer comprising silicon dioxide; and
a layer comprising an inorganic-organic hybrid polymer;
wherein the barrier layer has a layer thickness of from about 10.0 nm to 150 nm and is disposed between the first polymer layer and the second polymer layer, and
(ii) a cosmetic composition, which is packaged in the package and comprises:
a) an oxidizing compound comprising hydrogen peroxide and present in a total quantity of from about 1.5 to about 15% by weight, with respect to the total weight of the cosmetic composition,
b) a thickening agent comprising a copolymer of (meth)acrylic acid and acrylic acid ethyl ester and present in an amount of from about 0.40 to about 4.5% by weight, with respect to the total weight of the cosmetic composition;
c) an anionic surfactant comprising a sodium salt of C₁₂-C₁₄ alkylether sulphate with about 2 mol of ethylene oxide per mol of C₁₂-C₁₄ alkylether sulphate, wherein the anionic surfactant is present in a total quantity of from about 0.27 to about 1.0% by weight, with respect to the total weight of the cosmetic composition;
d) water present in a total quantity of from about 60 to about 97% by weight, with respect to the total weight of the cosmetic composition.

20. A cosmetic product for changing the natural colour of keratinous fibres comprising:
(i) a package, comprising a multilayer foil, which comprises:
a first polymer layer comprising polypropylene and having a layer thickness of from about 60.0 μm to about 90.0 μm,
a second polymer layer comprising polyethylene terephthalate and having a layer thickness of from about 10.0 μm to about 20.0 μm, and
a barrier layer comprising:
a layer comprising silicon dioxide; and
a layer comprising an inorganic-organic hybrid polymer;
wherein the barrier layer has a layer thickness of from about 10.0 nm to 150 nm and is disposed between the first polymer layer and the second polymer layer, and
(ii) a cosmetic composition, which is packaged in the package and consists of:
a) an oxidizing compound,
b) a thickening agent comprising a copolymer of (meth)acrylic acid and acrylic acid ethyl ester;
c) an anionic surfactant comprising a sodium salt of C₁₂-C₁₄ alkylether sulphate with about 2 mol of ethylene oxide per mol of C₁₂-C₁₄ alkylether sulphate;
d) water present in a total quantity of from about 60 to about 97% by weight, with respect to the total weight of the cosmetic composition;
e) an inorganic alkalizing agent; and
f) a mixture of acids comprising:
at least one of dipicolinic acid, disodium pyrophosphate, ethylenediamine tetraacetic acid, or salts thereof; and
1-hydroxyethane-1,1-diphosphonic acid.
